(19) United States
(12) Patent Application Publication
(10) Pub. No.: US 2016/0213492 A1
(43) Pub. Date: Jul. 28, 2016

(54) IMPACTOR ADAPTOR AND SCREW ASSEMBLY

(71) Applicant: Trigon International Corporation, Aurora, IL (US)

(72) Inventors: Gary Castello, Montgomery, IL (US); Nicholas Diaz, Yorkville, IL (US); Joseph Fenoglio, Naperville, IL (US); Robert J. Fenoglio, New Lenox, IL (US); Mark C. Galvin, Naperville, IL (US); James M. Walsh, Naperville, IL (US)

(21) Appl. No.: 15/006,623

(22) Filed: Jan. 26, 2016

Related U.S. Application Data
(60) Provisional application No. 62/108,316, filed on Jan. 27, 2015.

(51) Int. Cl. A61F 2/46

(52) U.S. Cl. CPC A61F 2/4609; A61F 2002/4624; A61F 2002/4628

(57) ABSTRACT

A prosthesis impactor assembly is provided having an impactor handle, an adaptor separate from the impactor handle, a connector rotatably secured within the adaptor, and a locking mechanism to lock the adaptor and connector to the impactor handle. The connector has a prosthesis connector member for connecting to the acetabular cup at one end, and a drive connector at an opposing end. The adaptor is received by a receiver of the impactor handle and is locked in place with the locking mechanism. The adaptor and impactor handle further have timing members to properly align the adaptor with respect to the impactor handle.

### CROSS-REFERENCE TO RELATED APPLICATIONS

This application claims the benefit of U.S. Provisional Patent Application No. 62/108,316, filed Jan. 27, 2015, which is expressly incorporated herein by reference and made a part hereof.

### FEDERALLY SPONSORED RESEARCH OR DEVELOPMENT

Not Applicable.

### TECHNICAL FIELD

The present invention relates generally to an adaptor and screw assembly, and more specifically to an acetabular cup impactor adaptor and cup screw assembly.

### BACKGROUND OF THE INVENTION

Acetabular cup impactor assemblies are well known in the art. While such impactor assemblies according to the prior art provide a number of advantages, they nevertheless have certain limitations. The present invention seeks to overcome certain of these limitations and other drawbacks of the prior art, and to provide new features not heretofore available. A full discussion of the features and advantages of the present invention is deferred to the following detailed description, which proceeds with reference to the accompanying drawings.

### SUMMARY

According to one embodiment, the disclosed subject technology relates to an acetabular cup impactor adaptor and cup screw assembly that is used to insert an acetabular cup into a patient. The adaptor and cup screw assembly is separate from the impactor housing/handle and this can be customizable to the specific acetabular cup to be inserted. For example, different acetabular cups may require different threads on the screw connector, different diameter of the connector, different depth of the threads, etc. Similarly, the acetabular cup may require a different configuration for the adaptor to engage the adaptor and for transferring force from the adaptor to the acetabular cup. Because the adaptor and the connector of the subject technology are interchangeable on the impactor housing, the present system can be used with any existing or to be designed acetabular cups.

The disclosed subject technology further relates to a prosthesis impactor assembly for inserting an acetabular cup comprising: an impactor handle; an adaptor having a central bore, the adaptor being separate from the impactor handle; a connector rotatably secured within the central bore of the adaptor, the connector having a first end and a second end, wherein the first end of the connector has a prosthesis connector member for connecting to the acetabular cup, and wherein the second end of the connector has a drive connector; a receiving assembly having a locking mechanism to removably secure the adaptor and connector to the impactor handle; and, a drive mechanism extending through a portion of the impactor handle and selectively engaging the drive connector of the connector.

The disclosed subject technology further relates to an adaptor that has a first end and a second end, wherein an impact head is provided at the first end of the adaptor, wherein an extension is provided at the second end of the adaptor, and wherein the central bore of the adaptor extends from the first end to the second end of the adaptor. In another embodiment, the extension has an external groove to receive a locking mechanism to assist in locking the adaptor to the impactor handle. In another embodiment, a radial flange extends from a perimeter of the adaptor to engage a shoulder of the acetabular cup, the radial flange having timing members to seat the adaptor to the acetabular cup.

The disclosed subject technology further relates to a connector member that extends out of the central bore of the adaptor to engage the acetabular cup. In one embodiment, the connector has a shoulder that engages a stop in the adaptor. Additionally, in another embodiment a retainer is provided to laterally fix the connector within the adaptor, but which still allows the connector to rotate within the adaptor.

The disclosed subject technology further relates to a locking mechanism that moves from a first position to a second position, the locking mechanism engaging the adaptor in the first position to axially lock the adaptor in the impactor handle, and the adaptor being removable from the impactor handle when the locking mechanism is in the second position.

The disclosed subject technology further relates to a receiving assembly that comprises an opening in the impactor handle for receiving a portion of the adaptor, a timing assembly for rotationally aligning the adaptor to the impactor handle, and a locking mechanism to removably secure the adaptor and connector to the impactor handle.

The disclosed subject technology further relates to a timing assembly that comprises a plurality of tapered ribs on the adaptor and a plurality of mating tapered ribs on the impactor handle. According to another embodiment, the timing assembly is provided on the adaptor and a mating timing assembly on the impactor handle.

The disclosed subject technology further relates to a locking mechanism that comprises a locking blade, a pivotable locking lever, and a locking spring. In one embodiment, the locking mechanism is connected to the impactor handle. Further, the locking blade may move linearly within the impactor handle in a direction transverse to the receiving opening of the impactor handle. In another embodiment, the locking blade has first and second arms that selectively engage the adaptor in the receiving opening to axially fix the adaptor in the impactor handle.

The disclosed subject technology further relates to an embodiment where the impactor handle has a receiving opening for accepting the adaptor and connector.

The disclosed subject technology further relates to a prosthesis impactor assembly for inserting an acetabular cup comprising: an impactor handle having a receiving opening; an adaptor that is separate from the impactor handle, the adaptor having a first end and a second end, wherein an impact head is provided at the first end of the adaptor, wherein an extension is provided at the second end of the adaptor to be received in the receiving opening of the impactor handle, and wherein the adaptor has a central bore that extends from the first end to the second end of the adaptor; and, a connector rotatably secured within the central bore of the adaptor, the connector having a first end and a second end, wherein the first end of the connector has a prosthesis connector member for connecting to the acetabular cup, wherein the connector member extends out of the central bore of the adaptor to engage the acetabular cup, and wherein the second end of the connector has a drive connector.

The disclosed subject technology further relates to a locking mechanism that is connected to the impactor handle to removably secure the adaptor and connector to the impactor handle, the locking mechanism comprising a locking blade, a pivotable locking lever, and a locking spring, wherein the locking blade moves linearly from a first position to a second position, the locking blade engaging the adaptor in the first position to axially lock the adaptor in the impactor handle, and the adaptor being removable from the impactor handle when the locking blade is in the second position.

The disclosed subject technology further relates to a drive mechanism extending through a portion of the impactor handle and selectively engaging the drive connector of the connector to rotate the connector.

The disclosed subject technology further relates to a plurality of tapered rib timing members on an exterior surface of the impactor handle, and mating tapered rib timing members on a mating surface of the adaptor for rotationally aligning the adaptor to the impactor handle.

The disclosed subject technology further relates to a prosthesis impactor assembly for inserting an acetabular cup comprising: an impactor handle having a receiver and a plurality of timing members adjacent the receiver; an adaptor that is separate from the impactor handle, the adaptor having a first end and a second end, wherein an impact head is provided at the first end of the adaptor, wherein the second end of the adaptor is received by the receiving of the impactor handle, and wherein the adaptor has a central bore that extends from the first end to the second end of the adaptor, the adaptor further having a plurality of mating timing members to mate with the timing members of the impactor handle; a connector rotatably secured within the central bore of the adaptor, the connector having a first end and a second end, wherein the first end of the connector has a prosthesis connector member for connecting to the acetabular cup, wherein the connector member extends out of the central bore of the adaptor to engage the acetabular cup, and wherein the second end of the connector has a drive connector; and, a locking mechanism connected to the impactor handle to removably secure the adaptor and connector to the impactor handle, the locking mechanism comprising a locking blade that engages the adaptor in a first position of the locking blade to axially lock the adaptor in the impactor handle, and the adaptor being removable from the impactor handle when the locking blade is in a second position.

It is understood that other embodiments and configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

To understand the present invention, it will now be described by way of example, with reference to the accompanying drawings in which:

FIG. 1 is a perspective view of one embodiment of an acetabular impactor assembly having an acetabular impactor adaptor and screw assembly and an adaptor locking mechanism, with the adaptor locking mechanism in the locked position. In a preferred embodiment the impactor assembly is used for inserting an acetabular cup prosthesis.

FIG. 2 is a side exploded perspective view of the acetabular cup impactor assembly of FIG. 1.

FIG. 3 is a side exploded perspective view from the rear of the acetabular cup impactor assembly of FIG. 1.

FIG. 4 is a side perspective view of the acetabular cup impactor assembly of FIG. 1 with the adaptor locking mechanism in the unlocked position.

FIG. 5 is a side view of the acetabular cup impactor assembly of FIG. 1 with the adaptor locking mechanism in the locked position.

FIG. 6 is a partial bottom perspective view of the acetabular cup impactor assembly of FIG. 1 with the adaptor locking mechanism in the locked position.

FIG. 7 is a partial bottom perspective view of the acetabular cup impactor assembly of FIG. 1 with the adaptor locking mechanism in the unlocked position.

FIG. 8 is a side view of a portion of the acetabular cup impactor assembly as shown in FIG. 5, with acetabular cup removed from the impactor adaptor and cup screw assembly and the adaptor locking mechanism in the locked position.

FIG. 9 is a side view of a portion of the acetabular cup impactor assembly as shown in FIG. 4, with acetabular cup removed from the impactor adaptor and cup screw assembly and the adaptor locking mechanism in the unlocked position.

FIG. 10 is a cross-sectional side view of a portion of the acetabular cup impactor assembly having the impactor adaptor and cup screw assembly connected to an acetabular cup as shown in FIG. 5, with the adaptor locking mechanism in the locked position.

FIG. 11 is a cross-sectional side view of a portion of the acetabular cup impactor assembly having the impactor adaptor and cup screw assembly connected to an acetabular cup as shown in FIG. 4, with the adaptor locking mechanism in the unlocked position.

FIG. 12 is a perspective view of one embodiment of the adaptor locking mechanism in the locked position.

FIG. 13 is a cross-sectional view of one embodiment of the acetabular cup impactor assembly showing a portion of the adaptor locking mechanism of FIG. 12 in the locked position.

FIG. 14 is a perspective view of one embodiment of the adaptor locking mechanism in the unlocked position.

FIG. 15 is a cross-sectional view of one embodiment of the acetabular cup impactor assembly showing a portion of the adaptor locking mechanism of FIG. 14 in the unlocked position.

FIG. 16 is a side cross-sectional view of another embodiment of an acetabular cup adaptor and cup screw assembly.

FIG. 17 is a side cross-sectional view of another embodiment of an acetabular cup adaptor and cup screw assembly.

FIG. 18 is a side cross-sectional view of another embodiment of an acetabular cup adaptor and cup screw assembly.

FIG. 19A is a side cross-sectional view of another embodiment of an acetabular cup adaptor and cup screw assembly.

FIG. 19B is a top cross-sectional view of the acetabular cup adaptor and cup screw assembly of FIG. 19A.

FIG. 20A is a rear perspective view of another embodiment of an acetabular cup adaptor and cup screw assembly.

FIG. 20B is a side cross-sectional view of the acetabular cup adaptor and cup screw assembly of FIG. 20A.

FIG. 21A is a rear perspective view of another embodiment of an acetabular cup adaptor and cup screw assembly.

FIG. 21B is a rear-perspective cross-sectional view of the acetabular cup adaptor and cup screw assembly of FIG. 21A.

FIG. 22 is an alternate embodiment of an acetabular cup adaptor and cup screw assembly on an alternate impactor housing.

### DETAILED DESCRIPTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

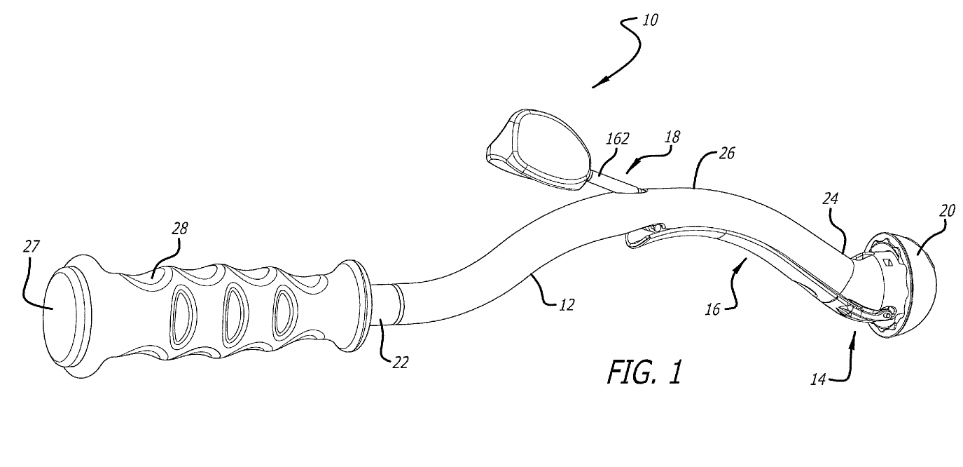

Referring now to the Figures, there is shown a variety of embodiments of a prosthesis impactor assembly 10 provided to aid a surgeon in controlling the installation of a prosthesis 20. In a preferred embodiment the prosthesis impactor assembly 10 is an acetabular cup impactor assembly 10 and the prosthesis is an acetabular cup prosthesis 20. As shown in FIGS. 1-3, in one embodiment the acetabular cup impactor assembly 10 generally comprises an impactor housing 12 or impactor handle 12, an acetabular cup impactor adaptor and cup screw assembly 14, an adaptor locking mechanism 16, and a drive mechanism 18 for the acetabular cup impactor adaptor and cup screw assembly 14. The acetabular cup impactor assembly 10 removably connects to the acetabular cup 20 to assist in securing the acetabular cup 20 within a patient.

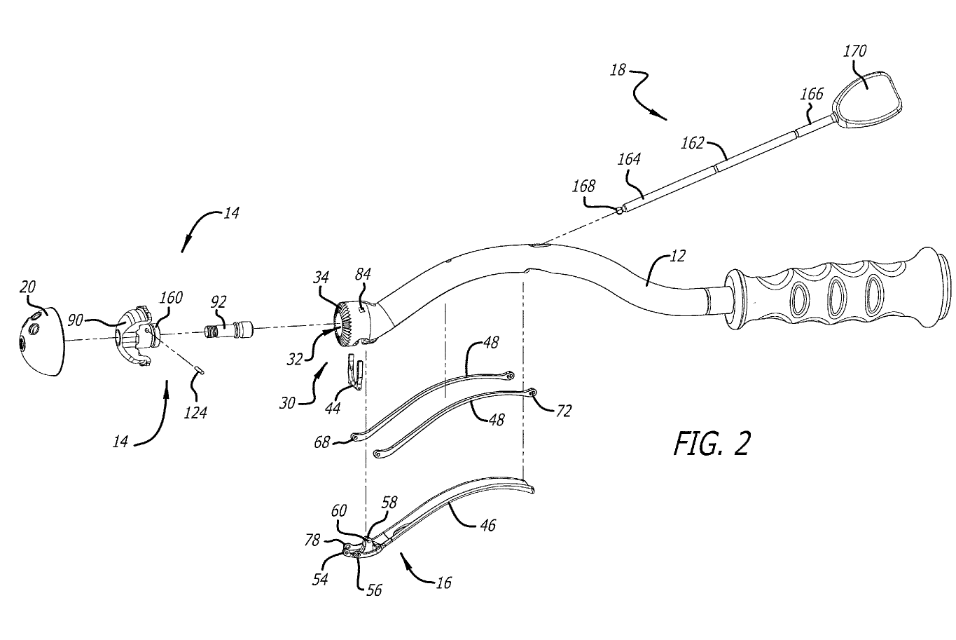

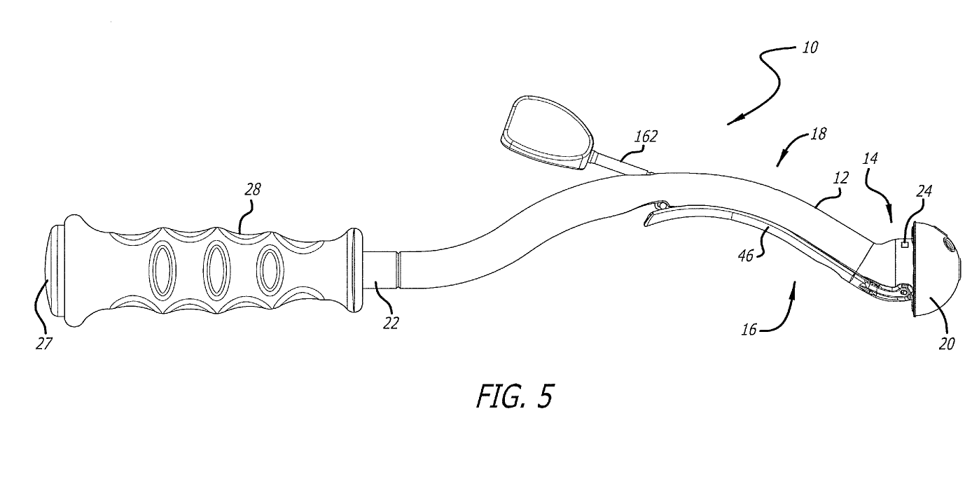

Referring to FIG. 2, the impactor housing 12 generally comprises a handle 12 having a distal end portion 22 spaced from a proximal end portion 24 with a length portion 26 residing therebetween. In one embodiment, not shown, the length portion 26 is generally straight. In an alternate embodiment, as best shown in FIGS. 2 and 5, among other figures, a portion of the length portion 26 is generally C-shaped to minimize the invasiveness of the surgery by less invasively clearing anatomical structures and tissue. A prosthesis, preferably an acetabular cup 20 is designed to be positioned adjacent the proximal end portion 24 of the impactor housing 12, preferably connected to the adaptor and cup screw assembly 14 at the proximal end portion 24 of the impactor housing 12. A strike plate 27 (see FIG. 1) resides at the distal end portion 22 of the impactor housing 12. The strike plate 27 is adapted to receive a force through a hammer by a surgeon or other clinician to set the prosthesis 20 in the patient. Additionally, a handle member 28 may also reside adjacent the distal end portion 22 of the impactor housing 12 to aid in gripping of the impactor assembly 10.

The proximal end portion 24 of the impactor housing 12 has a receiving assembly 30 for receiving the acetabular cup impactor adaptor and cup screw assembly 14. In one embodiment the receiving assembly 30 comprises a receiver 32, a timing member 34 and an adaptor locking mechanism 16.

Referring to FIGS. 2, 3 and 10, in one embodiment the receiver 32 comprises an opening 32, such as a female bore 32, which receives a male extension 36 extending from the adaptor and cup screw assembly 14 as described below. In alternate embodiments, not shown, the receiver 32 may be a male component that receives a female component of the adaptor and cup screw assembly 14, or some alternate mating mechanism.

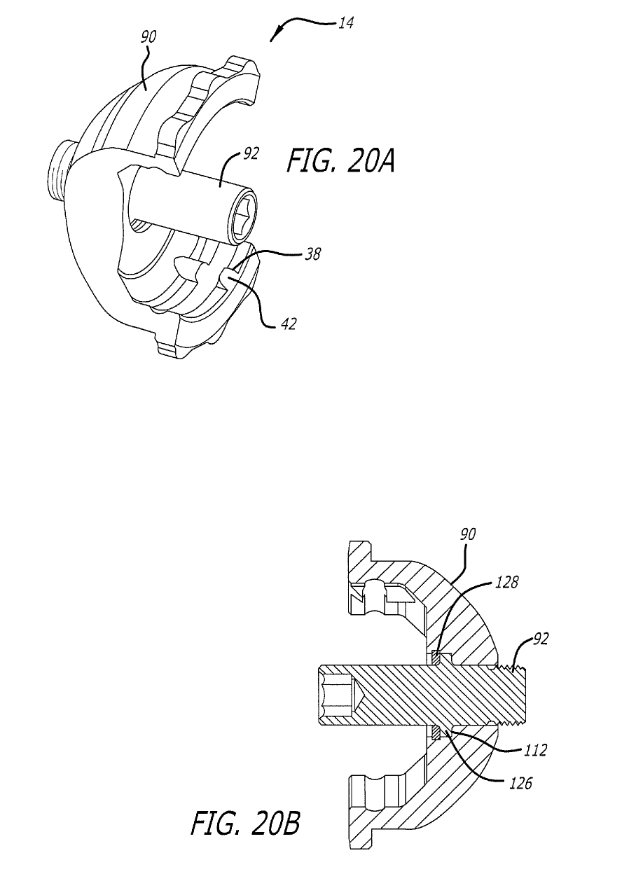

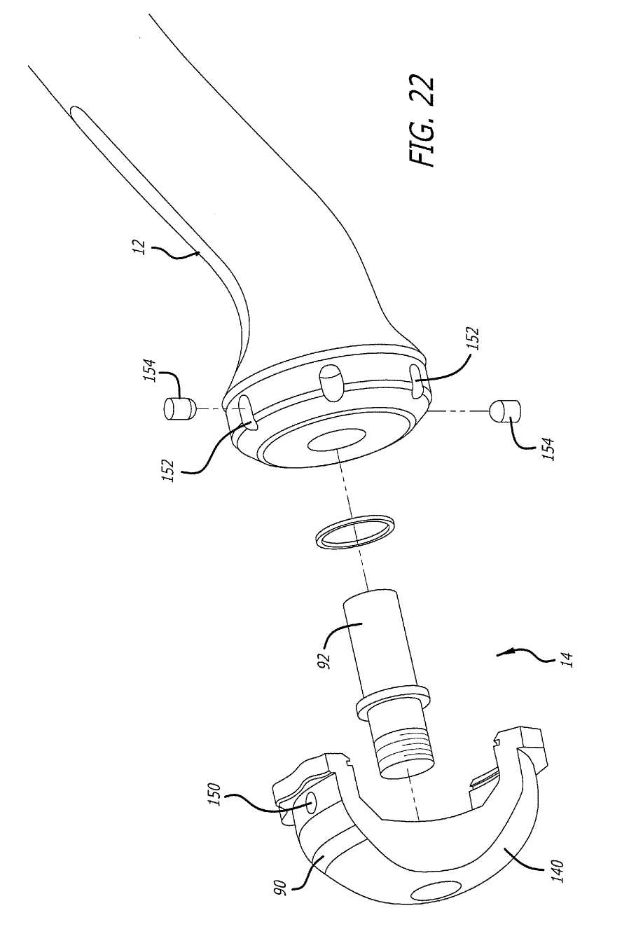

The timing member 34 of the receiving assembly 30 comprises a component that mates with the mating timing member 38 on the adaptor and cup screw assembly 14 to appropriately rotationally align the adaptor and cup screw assembly 14 to the impactor housing 12. As shown in FIGS. 2 and 16-19B, in one embodiment the timing member 34 on the impactor housing 12 comprises a plurality of tapered ribs 40 or a tapered spline 40 that mates with the mating timing member 38 on the adaptor and cup screw assembly 14. In this embodiment, where the timing member 34 on the impactor housing 12 is a plurality of tapered ribs 40, the timing member 38 on the adaptor and cup screw assembly 14 is likewise a plurality of mating tapered ribs 42. The mating tapered ribs 40 and 42 allow for custom rotational alignment of the adaptor and cup screw assembly 14 on the impactor housing 12. Additionally, the use of mating tapered ribs 40, 42 between the impactor housing 12 and the adaptor and cup screw assembly 14 provides to minimize the radial and axial play between the impactor housing 12 and the adaptor and cup screw assembly 14, which translates to less radial and axial play between the prosthetic cup 20 and the impactor assembly 10 once the adaptor and cup screw assembly 14 is joined to the impactor housing 12 and the prosthetic cup 20 is joined to the adaptor and cup screw assembly 14. Referring to FIGS. 20A and 20B, in an alternate embodiment the timing member 34 of the impactor housing 12 comprises one or more recesses (not shown) that mate with a corresponding one or more ribs 42, which act as the timing members 38 on the adaptor and cup screw assembly 14. In a further alternate embodiment as shown in FIG. 22, the adaptor and cup screw assembly 14 has a plurality of apertures 150 on a perimeter of the impact head 140, and the impactor housing 12 has a plurality of recesses 152. Pins 154 extend through the apertures 150 and into the recesses 152 so that the adaptor and cup screw assembly 14 can be rotationally aligned and connected to the impactor housing 12 at a variety of angular positions.

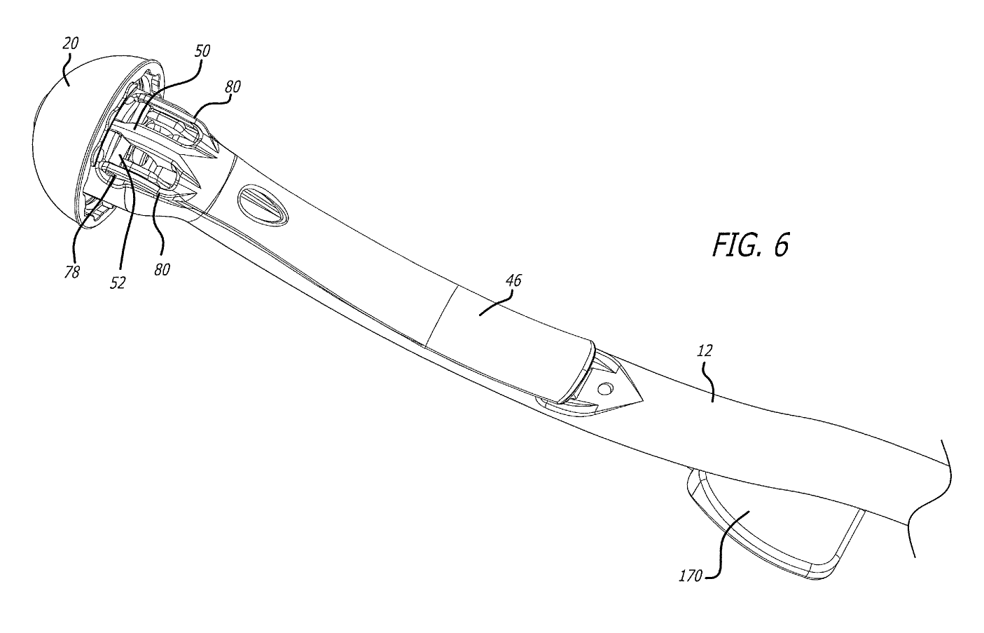

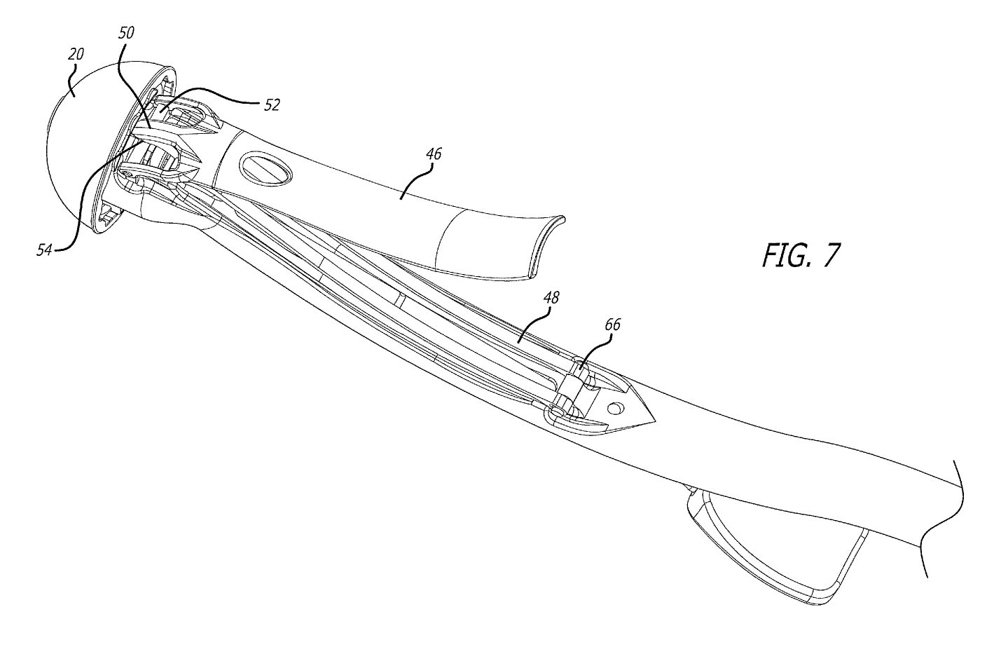

In a preferred embodiment the receiving assembly 30 also comprises an adaptor locking mechanism 16. The adaptor locking mechanism 16 operates to releasably secure in a locking and unlockable manner, the adaptor and cup screw assembly 14 to the impactor housing 12. As shown in FIGS. 2-3, 6-7 and 12-13, in one embodiment the adaptor locking mechanism 16 comprises a locking blade 44, a pivotable locking member 46 or lever 46, and a locking spring 48. In a preferred embodiment the adaptor locking mechanism 16 operates in a three-point over-center locking mechanism to lock the locking mechanism 16 in use. The over-center adaptor locking mechanism 16 provides positive clamping and tactile feedback to the user, and in most applications the operator will be able to lock the adaptor locking mechanism 16 with one hand. While the adaptor locking mechanism 16 in a preferred embodiment is an over-center locking mechanism, other locking mechanisms known in the art may also be used for locking the adaptor and cup screw assembly 14 to the impactor housing 12.

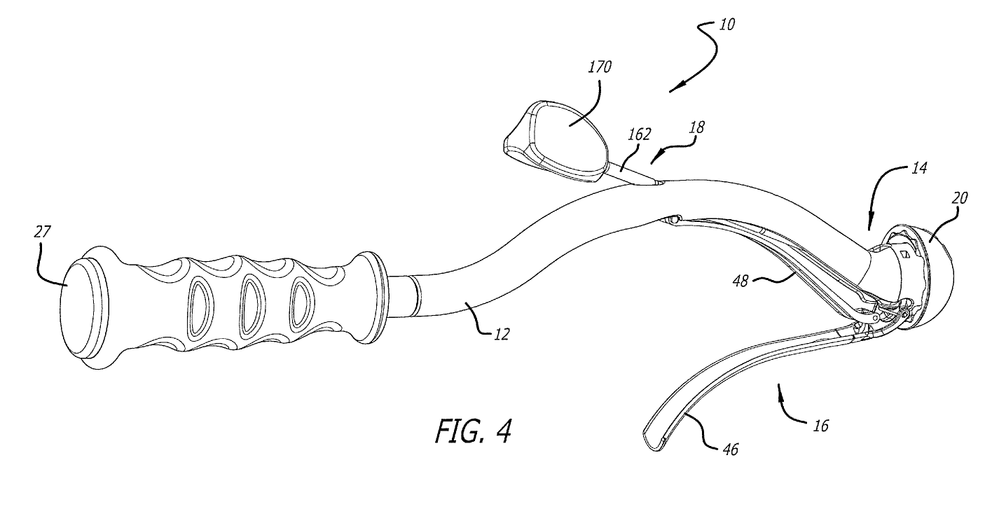

In one embodiment the adaptor locking mechanism 16, and specifically the locking member or lever 46 of the adaptor locking mechanism 16, is pivotally connected to the impactor housing 12 at a first pivot point 50 (see FIGS. 4, 6, 7 and 14). As shown in those figures, in one embodiment the impactor housing 12 has a shaft 52 or pin 52 toward the proximal end portion 24 of the impactor housing 12. Similarly, lever 46 has a bore 54 at a first end 56 of the lever 46 that receives the pin 52 to allow the lever 46 to pivot about the impactor housing 12. Pivot point 50 is thus referred to as the first pivot point (pivot 1). The lever 46 operates as a link in the toggle mechanism. Preferably, the lever 46 provides leverage for the user to operate the adaptor locking mechanism 16.

The lever 46 has a second pivot point 58 (pivot 2), which is eccentric with respect to pivot 1, and rotates about pivot 1. The second pivot point 58 is provided on an ear 60 extending from the lever 46. In a preferred embodiment, attached at, and rotating about the second pivot point 58 (pivot 2) is the locking spring 48, which is also referred to as an intermediate link. In a preferred embodiment the locking spring 48 operates as a type of leaf spring. As shown in FIG. 2, in one embodiment two locking springs 48 are provided, however, one may be adequate.

Each locking spring 48 has a first end 62 and a second end 64. The first end 62 of the intermediate link or locking spring 48 pivots about the second pivot point 58 (pivot 2) on the lever 46 and the second end 64 of the intermediate link or locking spring 48 pivots about a third pivot point 66 (pivot 3) on the impactor housing 12 (see FIGS. 4, 7, 10 and 11). In a preferred embodiment an aperture 68 is provided at the first end 62 of the locking spring 48 to pivotally connect to a shaft 70 extending from the ear 60 of the lever 46. Similarly, an aperture 72 is provided at the second end 64 of the locking spring 48 to pivotally connect to a shaft 72 extending from the impactor housing 12. To prevent binding, aperture 72 at the second end 64 of the locking spring 48 is slotted to the locking spring 48 to slightly move laterally with respect to the shaft 72 when the lever 46 is operated.

The locking spring 48, also referred to as the intermediate link 48, is manipulated or driven by rotation of the lever 46. The pivot points, points 1, 2 and 3 described above, are generally hinged connections.

In a preferred embodiment, the lever 46 is capable of locking in a closed position (see FIGS. 1, 5, 8 and 10 as examples). Lever 46 locks in position when pivot 2 passes “over center” through a line defined between pivot 1 and pivot 3. At the position where pivots 1, 2 and 3 are all in a line, compression of the locking spring 48 is greatest. By continuing to rotate the lever 46 about pivot point 1 toward the impactor housing 12, pivot point 2 is forced over the line causing a locking of the lever 46 and the locking spring 48. Forcing the pivot point 2 to be positioned over the line joining the three pivot points is referred to as “passing over center.” Thus, when pivot 2 passes over center in either direction it transitions from the locked position (see FIG. 1), to the unlocked position (see FIG. 4). Pivot point 2 cannot spontaneously pass over center in either direction without the operator's assistance. Thus, when the lever 46 is closed, i.e., when pivot 2 is passed over center toward the impactor housing 12, it locks the adaptor locking mechanism 16 in the locked position.

An alternate embodiment, not shown, the locking spring 48 and locking lever 46 are provided but the locking lever 46 is much shorter, and the spring 48 is concave away from the impactor housing 12. As expected, a variety of structural variations of the locking spring and locking lever are possible to still attain the desired over-center locking mechanism 16 of the present disclosure.

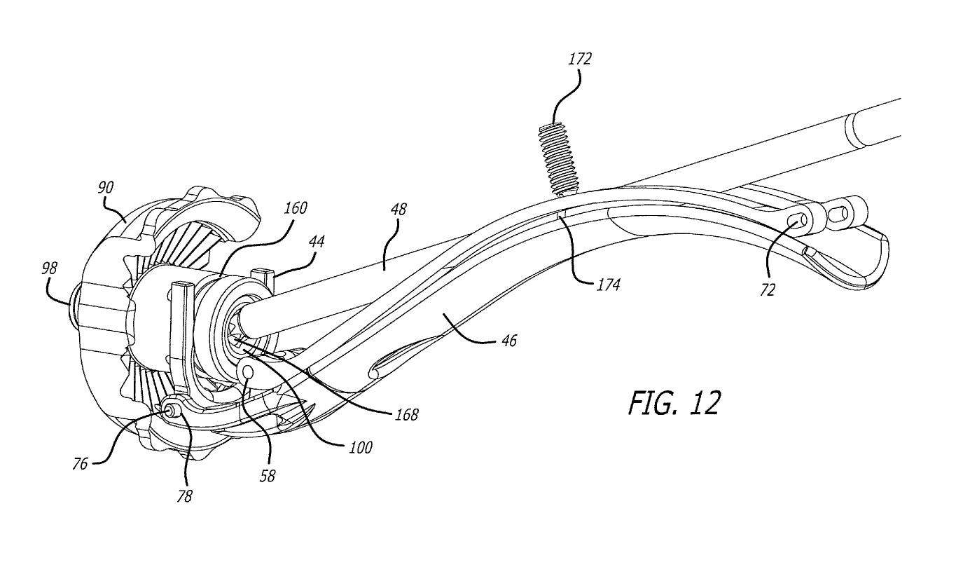

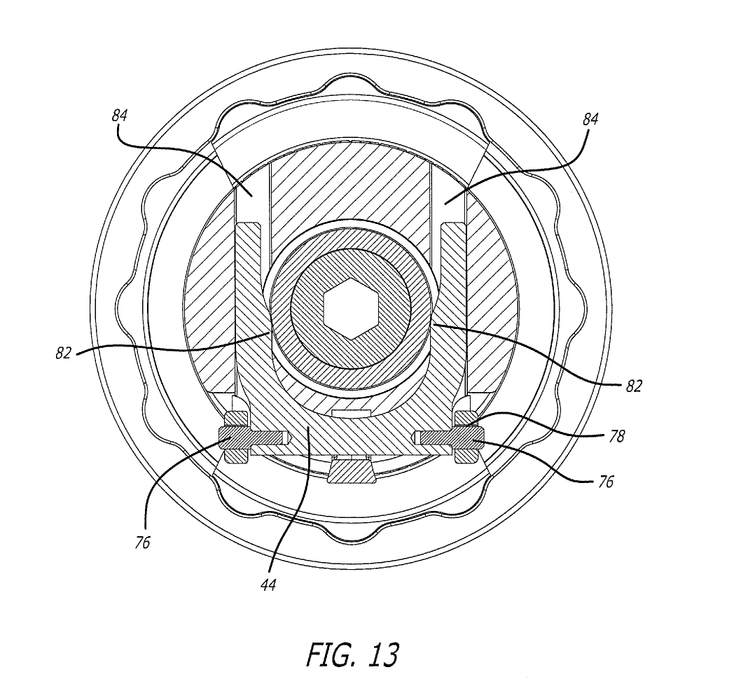

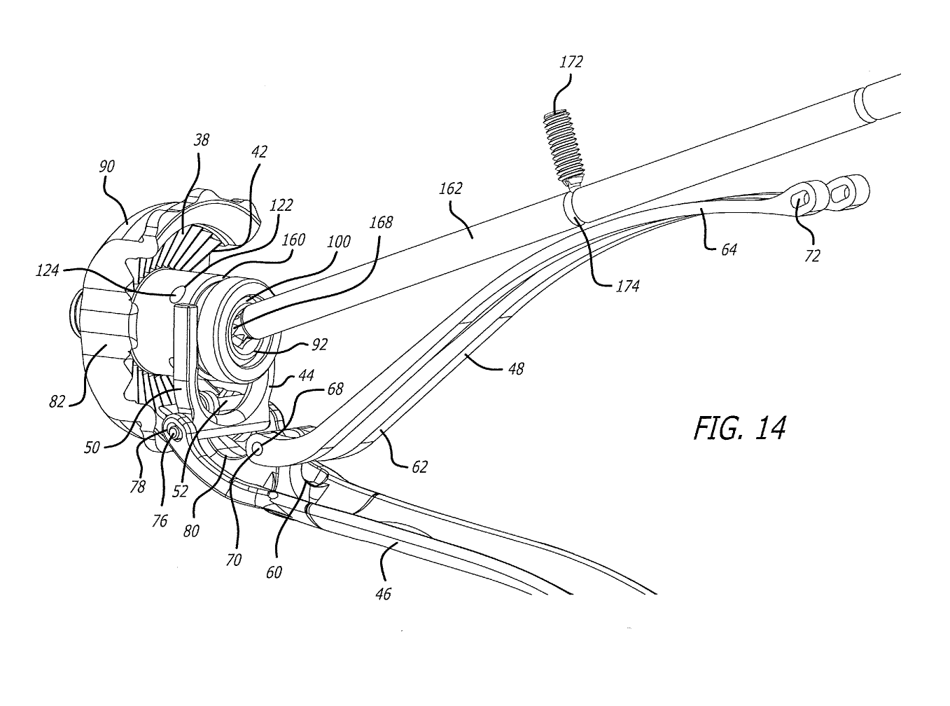

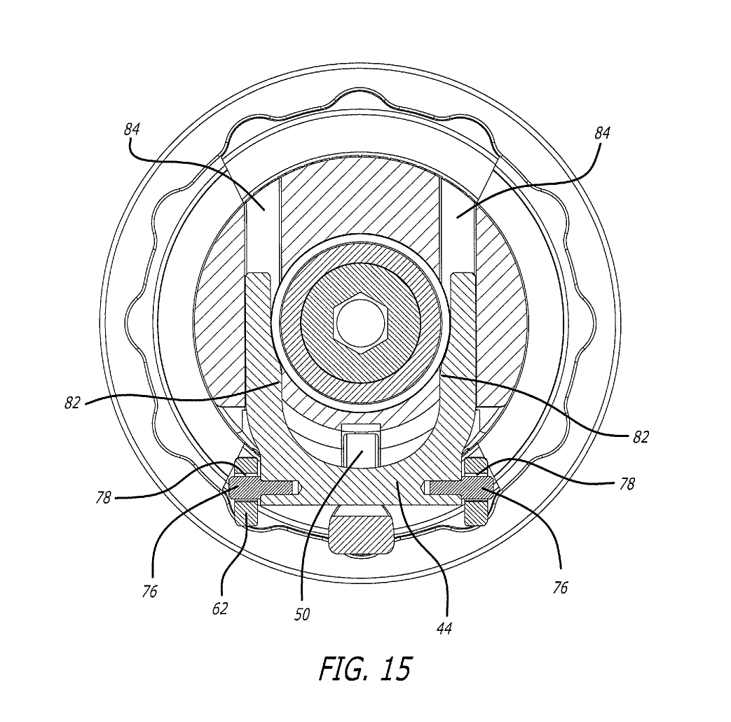

As shown in FIGS. 12 and 14, the locking blade 44 is connected to and driven by the locking member or lever 46. In a preferred embodiment, a pair of protrusions 76 extends from the locking blade 44 and are received by and mate with apertures 78 extending from arms 80 of the lever 46. In a preferred embodiment, because the lever 46 preferably undergoes rotational or pivotal movement about pivot point 1, and since the locking blade 44 preferably undergoes linear movement as will be described herein, the apertures 78 are slotted to prevent binding of the locking blade 44 during movement of the lever 46 between the unlocked position (see FIGS. 14 and 15) and the locked position (see FIGS. 12 and 13). In one embodiment the locking blade 44 has first and second arms 82 which form a U-shaped configuration as shown in FIGS. 12-15. The arms 82 are also preferably tapered to allow for tolerance differences. The first and second arms 82 move linearly within rectangular openings 84 in the impactor housing 12 (see openings 84 in FIGS. 2, 3, 8 and 9), and are provided to mate with the adaptor and cup screw assembly 14 to lock the adaptor and cup screw assembly 14 to the impactor housing 12. As shown in FIG. 13, when the arms 82 are in the locked position the adaptor and cup screw assembly 14 is locked in place, and as shown in FIG. 15, when the arms 82 are in the unlocked position the adaptor and cup screw assembly is unlocked axially and is free to be removed from the impactor housing 12.

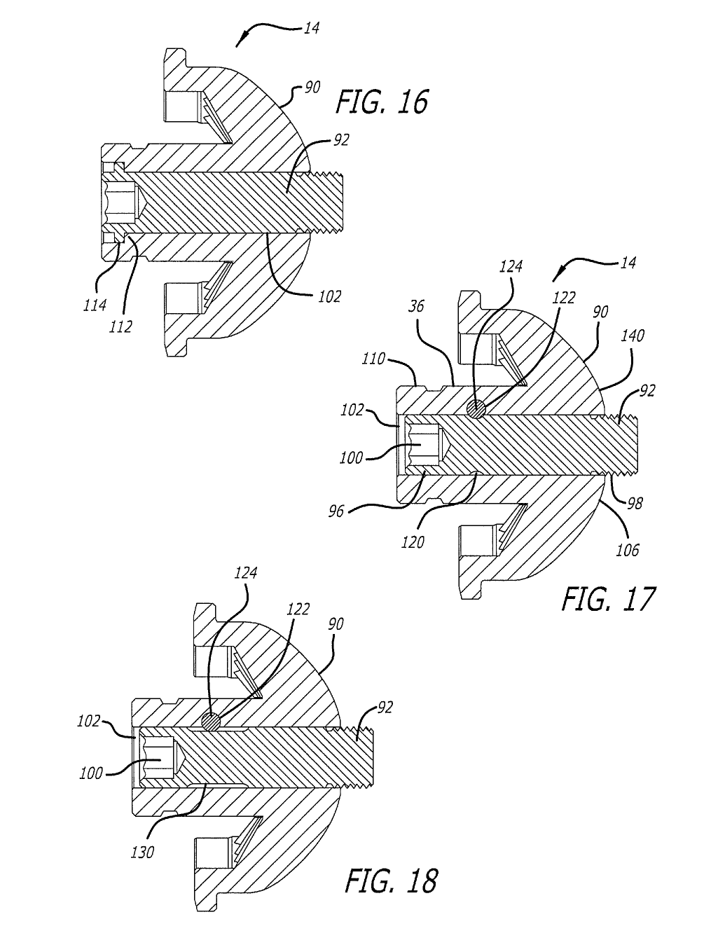

Referring to FIGS. 2-3, 10-12, 14 and 16-21B, in different embodiments the acetabular cup impactor adaptor and cup screw assembly 14 generally comprises an adaptor 90 and a prosthesis connector 92. In a preferred embodiment the prosthesis connector 92 is a cylindrical screw component 92. Further, in a preferred embodiment the prosthesis connector 92 is connected within the adaptor 90 in a rotational but linearly fixed manner through a variety of possible connections, several of which are shown in FIGS. 16-21B, to create the adaptor and cup screw assembly 14, however, in alternate embodiments the prosthesis connector 92 may be removable from the adaptor 90. Additionally, while the prosthesis connector 92 is said, in one embodiment, to be fixedly connected within the adaptor 90, the prosthesis connector 92 is generally able to rotate within the adaptor 90, but is preferably axially or laterally restricted or prevented from removal from the adaptor 90. It is understood that a variety of adaptors 90 and connectors 92 may be utilized with the same impactor housing 12. Thus, the adaptor 90 and connector 92 are designed to fit for the specific customer's prosthesis 20, while the same impactor housing 12 can always be used. This allows this system to be fully customizable for any prosthesis 20.

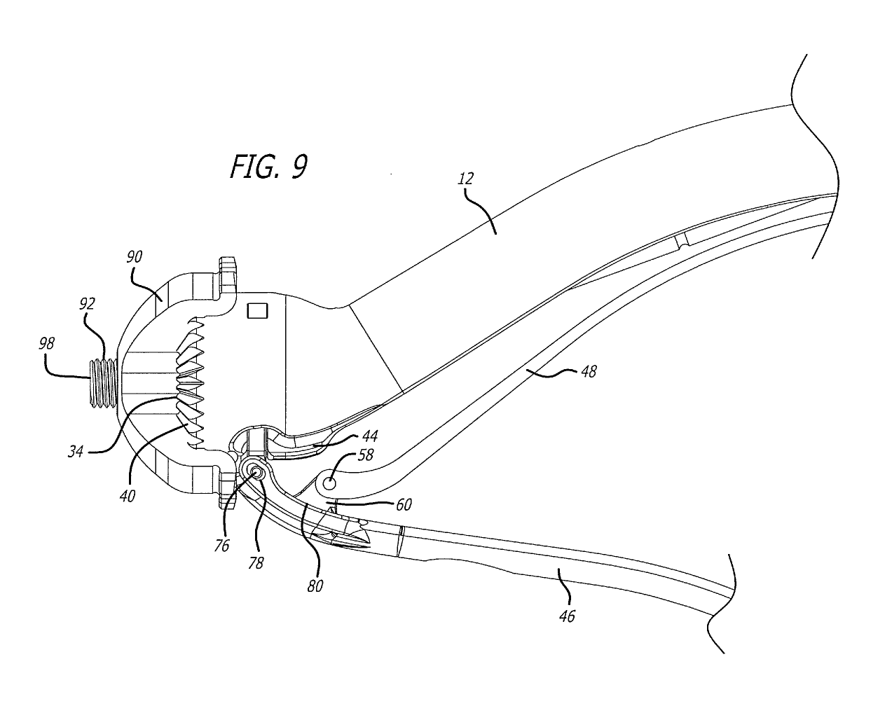

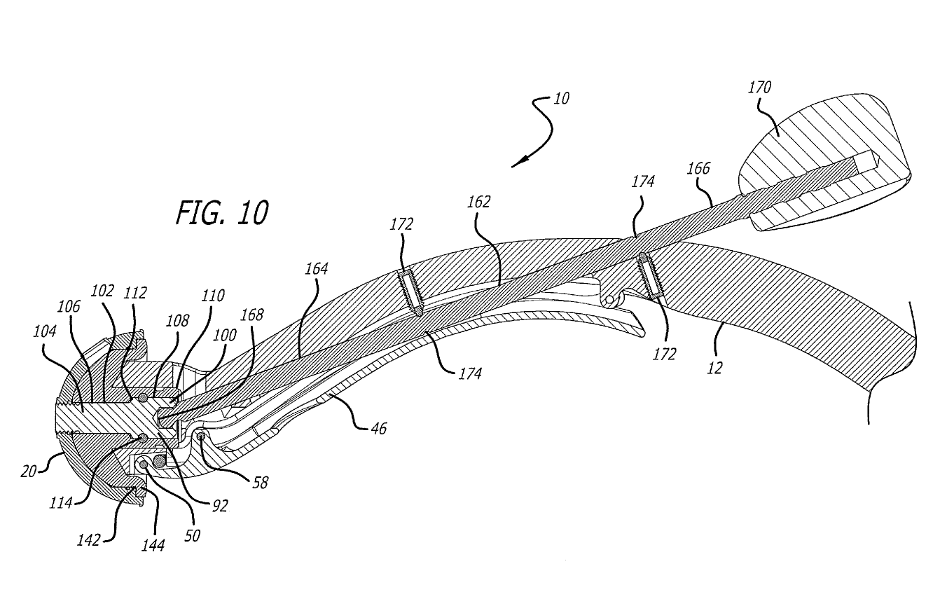

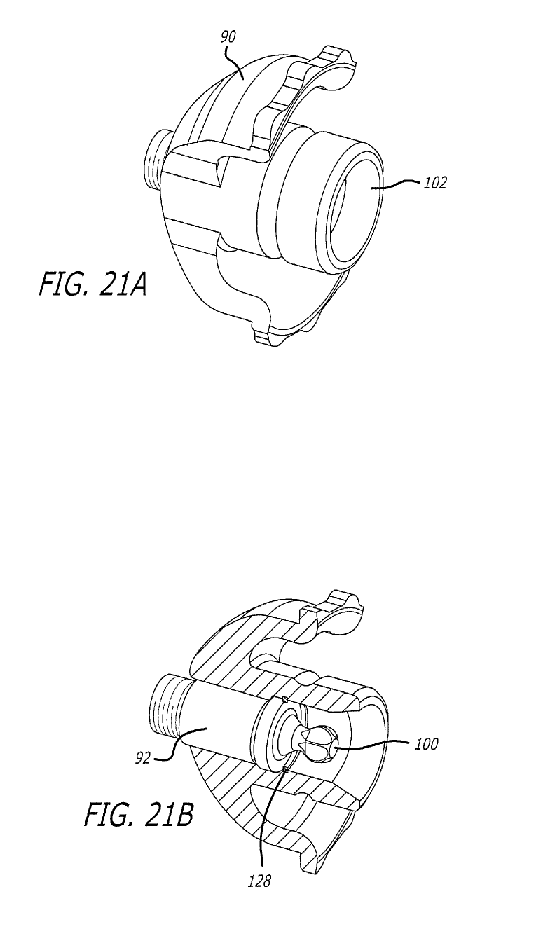

In one embodiment the prosthesis connector 92 has a first end 94 and a second end 96. A prosthesis connector member 98, such as threads 98, is provided at the first end 94 of the prosthesis connector 92, and a drive connector 100 is provided at the second end 96 of the prosthesis connector 92. In one embodiment, the drive connector 100 is a receiver for the drive mechanism 18 for the acetabular cup impactor adaptor and cup screw assembly 14. One preferable type of drive connector 100 receiver is a female hexagonal receiver for receiving a male hexagonal member at the end of the drive mechanism 18 (see, for example, FIGS. 10 and 17). Alternately, the drive connector 100 receiver may be a male hexagonal member for engaging a female hexagonal member at the end of the drive mechanism 18 (see, for example, FIGS. 21A and 21B). While a hexagonal connection is described, alternate mating mechanisms may be utilized without departing from the scope of the present disclosure.

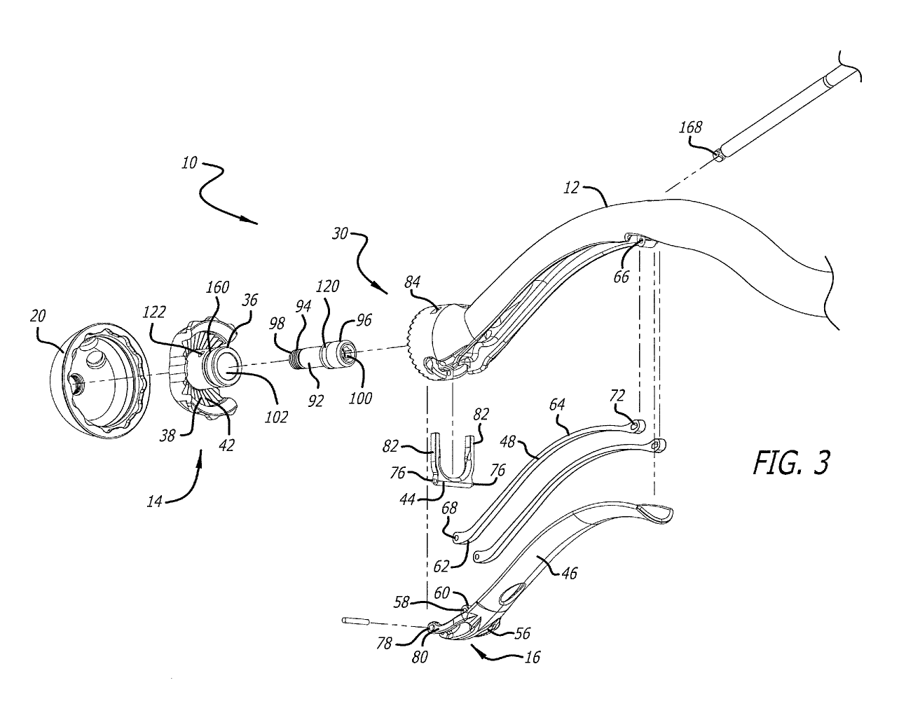

As shown in FIGS. 2-3, 10-12, 14 and 16-21B, the adaptor 90 preferably has a central bore 102 for receiving and rotationally housing the prosthesis connector 92. In various embodiments, the central bore 102 has a first bore diameter 104 adjacent a first end 106 of the adaptor 90 and a second bore diameter 108, generally a larger diameter, adjacent a second end 110 of the adaptor 90. The joint between the first bore diameter and the second bore diameter provides a stop 112 that engages a shoulder 114 on various embodiments of the prosthesis connector 92 (see FIGS. 3, 10, 17, 20A-20B and 21A-21B). In alternate embodiments, however, the central bore 102 may have a generally continuous bore diameter, such as shown in FIGS. 16, 18 and 19A-19B.

Referring to FIG. 17, the prosthesis connector 92 has a generally cylindrical body with a prosthesis connection member 98, i.e., threads 98, at a first end 94 thereof, and a drive connector 100, i.e., a female hexagonal receiver 100 at the second end 96 thereof. The prosthesis connector 92 has an annular groove 120 about its circumference. The adaptor 90 has a central bore 102 with a generally continuous bore diameter. The adaptor 90 also has an opening 122 through its male extension 36 that intersects the central bore 102. The opening 122 is offset from the axis of the central bore 102. The prosthesis connector 92 is inserted into the central bore 102 of the adaptor 90 and the annular groove 120 is aligned with the opening 122 in the adaptor 90. A pin 124 is inserted into the opening 122 and engages the annular groove 120 to axially fix the prosthesis connector 92 within the central bore 102 of the adaptor 90, however, the prosthesis connector 92 is able to freely rotate within the central bore 102. In an alternate embodiment the adaptor 90 may have a second opposing opening (not shown) to receive a second pin for further axially fixing the prosthesis connector 92 within the adaptor 90.

The prosthesis connector 92 in the embodiment shown in FIGS. 2 and 10 is similar to the prosthesis connector 92 shown in FIG. 17, however, the prosthesis connector 92 shown in FIGS. 2 and 10 also includes a shoulder 114 that engages a stop 112 within the central bore 102 of the adaptor 90 of this embodiment.

Referring to FIGS. 16, 20A-20B and 21A-21B, the prosthesis connector 92 has a generally cylindrical body with a prosthesis connection member 98, i.e., threads 98, at a first end 94 thereof, and a drive connector 100, i.e., a female hexagonal receiver 100 at the second end 96 thereof in the embodiment of FIGS. 16 and 20A-20B, and a male ball hexagonal receiver 100 at the second end 96 thereof in the embodiment of FIGS. 21A-21B. The prosthesis connector 92 in FIGS. 16, 20A-20B and 21A-21B has an annular flange 126 about its circumference that engages a stop 112 within the central bore 102 of the adaptor 90 of this embodiment. A fixing member 128, such as a bushing, washer, clip, etc., is fixed within the central bore 102 to axially retain the annular flange 126 against the stop 112 and axially fix the prosthesis connector 92 within the adaptor 90. The fixing member 128 may be fixed by a variety of methods, including, but not limited to pressure fit or welding. Accordingly, the prosthesis connector 92 is axially fixed but able to freely rotate within the central bore 102.

Referring to FIG. 18, the prosthesis connector 92 has a generally cylindrical body with a prosthesis connection member 98, i.e., threads 98, at a first end 94 thereof, and a drive connector 100, i.e., a female hexagonal receiver 100 at the second end 96 thereof. The prosthesis connector 92 has a wide annular groove 130 about its circumference. The adaptor 90 has a central bore 102 with a generally continuous bore diameter. The adaptor 90 also has an opening 122 through its male extension 36 that intersects the central bore 102. The opening 122 is offset from the axis of the central bore 102. The prosthesis connector 92 is inserted into the central bore 102 of the adaptor 90 and the wide annular groove 130 is aligned with the opening 122 in the adaptor 90. A pin 124 is inserted into the opening 122 and engages the wide annular groove 130 to axially retain the prosthesis connector 92 within the central bore 102 of the adaptor 90, however, the prosthesis connector 92 is able to freely rotate within the central bore 102.

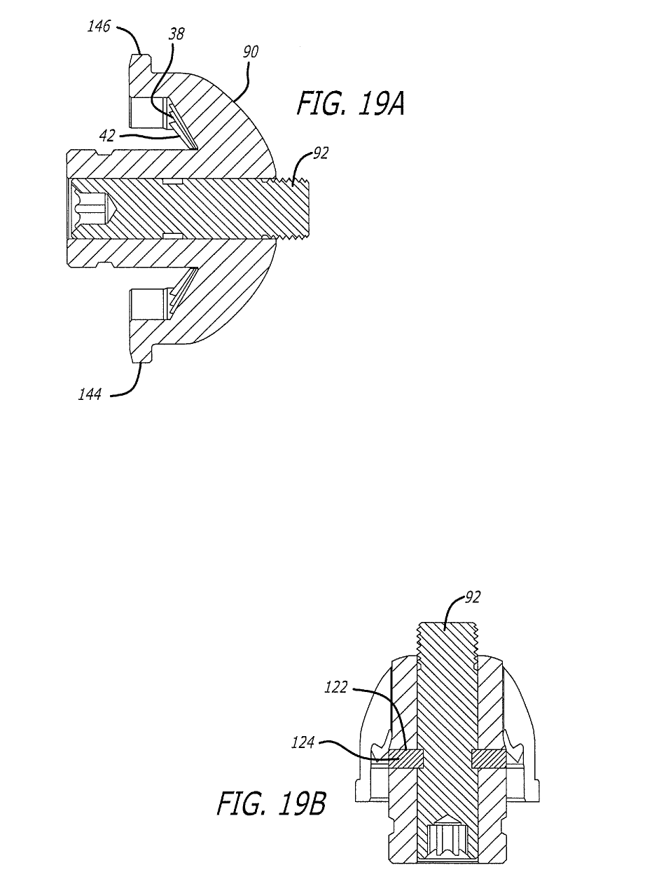

Referring to FIGS. 19A and 19B, the prosthesis connector 92 has a generally cylindrical body with a prosthesis connection member 98, i.e., threads 98, at a first end 94 thereof, and a drive connector 100, i.e., a female hexagonal receiver 100 at the second end 96 thereof. The prosthesis connector 92 has an annular groove 120 about its circumference. The adaptor 90 has a central bore 102 with a generally continuous bore diameter. The adaptor 90 also has an opening 122 through its male extension 36 that intersects the central bore 102. In this embodiment, the opening 122 is in-line with the axis of the central bore 102, unlike the opening 122 in the embodiment of FIG. 17. The prosthesis connector 92 is inserted into the central bore 102 of the adaptor 90 and the annular groove 120 is aligned with the opening 122 in the adaptor 90. A pin 124 is inserted into the opening 122 and engages the annular groove 120 to axially fix the prosthesis connector 92 within the central bore 102 of the adaptor 90, however, the prosthesis connector 92 is able to freely rotate within the central bore 102. In an alternate embodiment the adaptor 90 may have a second opposing opening to receive a second pin for further axially fixing the prosthesis connector 92 within the adaptor 90.

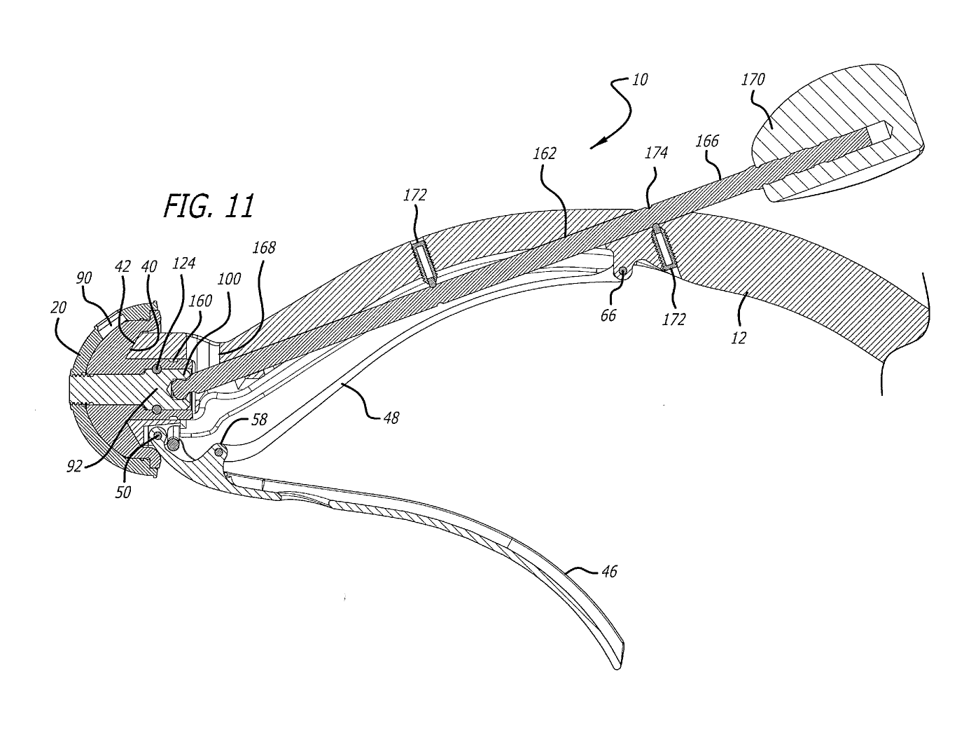

As explained above, in one embodiment the adaptor 90 preferably is a component that connects to the prosthesis 20, such as an acetabular cup prosthesis 20. The adaptor 90 comprises a male extension portion 36 and an impact head 140, which is also referred to as the prosthesis facing portion 140. The adaptor 90 has a first end 106 and a second end 110, with the impact head 140 being located at the first end 106 of the adaptor 90 and the male extension portion 36 extending from the impact head 140 and being located toward the second end 110 of the adaptor 90. A central bore 102 for receiving and rotationally housing the prosthesis connector 92 generally extends from the first end 106 to the second end 110 of the adaptor 90. At the first end 106 the central bore 102 is partially housed with the impact head 140, and at the second end 110 the central bore 102 is partially housed in the male extension portion 36 of the adaptor 90. Referring to FIGS. 10 and 11, in one embodiment the male extension portion 36 of the adaptor 90 is received in the female bore 32 of the receiving assembly 30 of the impactor housing 12. Further, as show in the figures the prosthesis connector 92 extends out of the central bore 102 in the adaptor 90 and beyond the first end 106 of the adaptor 90 to engage and retain the prosthesis 20.

At the first end 106 of the adaptor 90, when the prosthesis 20 is an acetabular cup 20, a portion of the outer surface 142 of the impact head 140 is generally spherical in shape. In one embodiment the impact head 140 portion from the front end appears to have an X-shape (see FIGS. 16-21B). A radial flange 144 extends from the perimeter of the impact head 140 to engage a shoulder 142 in the interior of the prosthesis 20 as shown in FIGS. 10 and 11. The radial flange 144 may also have a unique perimetral shape 146, such as a series of frustoconical members 146, which mates with like shape on the prosthesis 20 to properly seat and time the adaptor 90 to the prosthesis 20.

As explained above, in one embodiment the impact head 140 may contain a timing member 38 for rotationally aligning the adaptor and cup screw assembly 14 with the impactor housing 12. In one embodiment, the timing member 38 may comprise a plurality of tapered ribs 42 on the inner surface 148 of the impact head 140 as shown in FIGS. 2, 3, 8, 9 and 16. The tapered ribs 42 on the adaptor 90 engage mating tapered ribs 40 on the impactor housing 12. The mating of the tapered ribs 40 and 42 allows for custom rotational alignment of the adaptor and cup screw assembly 14 on the impactor housing 12. Additionally, the use of mating tapered ribs 40, 42 between the impactor housing 12 and the adaptor and cup screw assembly 14 provides to minimize the radial and axial play between the impactor housing 12 and the adaptor and cup screw assembly 14, which translates to less radial and axial play between the prosthetic cup 20 and the impactor assembly 10 once the adaptor and cup screw assembly 14 is joined to the prosthetic cup 20. In an alternate embodiment, such as shown in FIGS. 20A and 20B, an alternate timing member 34 of the impactor housing 12 comprises one or more recesses (not shown) that mate with a corresponding one or more ribs 42, which act as the timing members 38 on the adaptor and cup screw assembly 14. A further alternate embodiment of the timing member is shown in FIG. 22. In that embodiment the adaptor and cup screw assembly 14 has a plurality of apertures 150 on a perimeter of the impact head 140, and the impactor housing 12 has a plurality of recesses 152. Pins 154 extend through the apertures 150 and into the recesses 152 so that the adaptor and cup screw assembly 14 can be rotationally aligned and connected to the impactor housing 12 at a variety of angular positions.

Referring to FIGS. 2, 3, 10 and 12-15, in one embodiment the adaptor and cup screw assembly 14 also has a means for being secured to the impactor housing 12. In one embodiment the adaptor locking mechanism 16 is utilized to removably secure the adaptor and cup screw assembly 14 to the impactor housing 12. Accordingly, in that embodiment the male extension portion 36 of the adaptor 90 has a groove 160 in its outer surface for engagement with the adaptor locking mechanism 16. Referring to FIGS. 14 and 15, in this embodiment the locking lever 46 for the adaptor locking mechanism 16 is in the unlocked position. As shown in FIG. 15, when the locking lever 46 is in the unlocked position the locking blade 44 does not engage the groove 160 in the adaptor 90. But, referring to FIGS. 12 and 13, and specifically FIG. 13, when the locking lever 46 is transitioned to the locked position, each leg of the locking blade 44 traverses linearly and upwardly within the respective rectangular openings 84 in the impactor housing 12 and engages the groove 160 in the adaptor 90 (see FIG. 13). When the locking blades 44 engage the groove 160 in the adaptor 90 the adaptor 90 is axially fixed within the receiver 32 of the receiving assembly 30 of the impactor housing 12 and the adaptor 90 cannot move axially. To release the adaptor and cup screw assembly 14 from the impactor housing 12, such as to replace it with a different adaptor and cup screw assembly 14 for a different prosthesis 20, the user would simply transition the locking lever 46 to the unlocked position (see FIGS. 14 and 15), and the adaptor and cup screw assembly 14 would be free to be removed from the impactor housing 12.

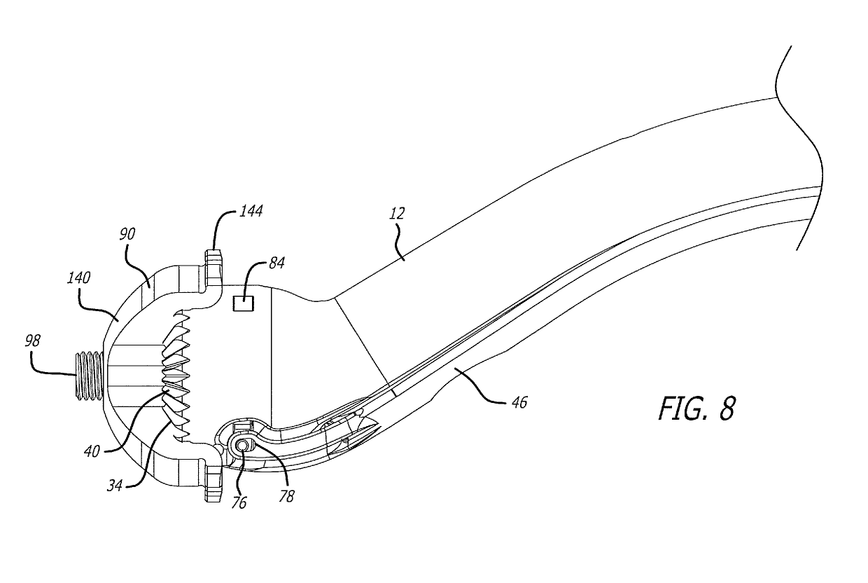

As shown in FIG. 8, when the adaptor locking mechanism 16 is in the locked position and the adaptor and cup screw assembly 14 is secured in place to the impactor housing 12, the operator is free to secure the prosthesis 20 to the impactor assembly 10. Referring to FIG. 8, in this position the connection member 98 (e.g., threads) of the prosthesis connector 92 (e.g., screw) extend out of the adaptor 90 and the connector 92 is adapted to be rotated. The operator can place a prosthesis 20, such as an acetabular cup 20, over the adaptor 90 and align the threaded opening 162 of the acetabular cup 20 (see FIG. 10) with the threads 98 of the screw 92.

To secure the prosthesis 20 to the adaptor and cup screw assembly 14, the operator uses the drive mechanism 18 of the impactor assembly 10. The drive mechanism 18 comprises a drive shaft 162 having a first end 164 and a second end 166. In this embodiment, the first end 164 of the drive shaft 162 has a male hexagonal ball member 168 and the second end 166 of the drive shaft 162 has a handle 170. The first end 164 of the drive shaft 162 is insertable into an opening in the impactor housing 12 to engage the prosthesis connector 92 as shown in FIG. 10. Two set screws 172, spring loaded plungers 172 or the like are used to hold the drive shaft 162 in place linearly, but also to allow the drive shaft 162 to be rotated. The drive shaft 162 may have grooves 174 to seat the set screws 172. To remove the drive shaft 162 from the impactor assembly 10 one would merely loosen the set screws 172 and pull the drive shaft 162 from the impactor housing 12.

When the drive shaft 162 is properly set in place in the impactor housing 12, the first end 164 of the drive shaft 162 engages the drive connector 100 of the prosthesis connector 92 as shown in FIG. 10. In this manner when an operator turns the handle 170 on the drive shaft 162, the drive shaft rotates the prosthesis connector 92. When the prosthesis connector 92 is rotated, the threads 98 on the prosthesis connector 92 will engage the threads in the threaded opening 162 of the acetabular cup 20. By further rotating the drive shaft 162 the acetabular cup 20 will be pulled toward and fully connected to the prosthesis impactor assembly 10.

Once the prosthesis 20 is connected to the prosthesis impactor assembly 10 the operator can insert the prosthesis 20 into a patient. After the prosthesis is inserted in a patient, the operator can unscrew the prosthesis from the prosthesis impactor assembly through reverse rotation of the drive shaft 162 and the prosthesis will become disengaged from the prosthesis impactor assembly 10.

Several alternative embodiments and examples have been described and illustrated herein. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. Additionally, the terms “first,” “second,” “third,” and “fourth” as used herein are intended for illustrative purposes only and do not limit the embodiments in any way. Further, the term “plurality” as used herein indicates any number greater than one, either disjunctively or conjunctively, as necessary, up to an infinite number.

It will be understood that the invention may be embodied in other specific forms without departing from the spirit or central characteristics thereof. The present examples and embodiments, therefore, are to be considered in all respects as illustrative and not restrictive, and the invention is not to be limited to the details given herein. Accordingly, while the specific embodiments have been illustrated and described, numerous modifications come to mind without significantly departing from the spirit of the invention and the scope of protection is only limited by the scope of the accompanying Claims.

What is claimed is:

1. A prosthesis impactor assembly for inserting an acetabular cup comprising:
an impactor handle;
an adaptor having a central bore, the adaptor being separate from the impactor handle;
a connector rotatably secured within the central bore of the adaptor, the connector having a first end and a second end, wherein the first end of the connector has a prosthesis connector member for connecting to the acetabular cup, and wherein the second end of the connector has a drive connector;
a receiving assembly having a locking mechanism to removably secure the adaptor and connector to the impactor handle; and,
a drive mechanism extending through a portion of the impactor handle and selectively engaging the drive connector of the connector.

2. The prosthesis impactor assembly of claim 1, wherein the connector member extends out of the central bore of the adaptor to engage the acetabular cup.

3. The prosthesis impactor assembly of claim 1, wherein the connector has a shoulder that engages a stop in the adaptor.

4. The prosthesis impactor assembly of claim 1, further comprising a retainer to laterally fix the connector within the adaptor but which still allows the connector to rotate within the adaptor.

5. The prosthesis impactor assembly of claim 1, wherein the locking mechanism moves from a first position to a second position, the locking mechanism engaging the adaptor in the first position to axially lock the adaptor in the impactor handle, and the adaptor being removable from the impactor handle when the locking mechanism is in the second position.

6. The prosthesis impactor assembly of claim 1, wherein the receiving assembly comprises an opening in the impactor handle for receiving a portion of the adaptor, a timing assembly for rotationally aligning the adaptor to the impactor handle, and the locking mechanism to removably secure the adaptor and connector to the impactor handle.

7. The prosthesis impactor assembly of claim 6, wherein the timing assembly comprises a plurality of tapered ribs on the adaptor and a plurality of mating tapered ribs on the impactor handle.

8. The prosthesis impactor assembly of claim 6, wherein the locking mechanism comprises a locking blade, a pivotable locking lever, and a locking spring, and wherein the locking mechanism is connected to the impactor handle.

9. The prosthesis impactor assembly of claim 1, wherein the impactor handle has a receiving opening for accepting the adaptor and connector.

10. The prosthesis impactor assembly of claim 9, wherein the locking mechanism comprises a locking blade that moves linearly within the impactor handle in a direction transverse to the receiving opening of the impactor handle.

11. The prosthesis impactor assembly of claim 10, wherein the locking blade has first and second arms that selectively engage the adaptor in the receiving opening to axially fix the adaptor in the impactor handle.

12. The prosthesis impactor assembly of claim 1, further comprising a timing assembly on the adaptor and a mating timing assembly on the impactor handle.

13. The prosthesis impactor assembly of claim 1, wherein the adaptor has a first end and a second end, wherein an impact head is provided at the first end of the adaptor, wherein an extension is provided at the second end of the adaptor, and wherein the central bore of the adaptor extends from the first end to the second end of the adaptor.

14. The prosthesis impactor assembly of claim 13, wherein the extension has an external groove to receive the locking mechanism to assist in locking the adaptor to the impactor handle.

15. The prosthesis impactor assembly of claim 13, further comprising a radial flange extending from a perimeter of the adaptor to engage a shoulder of the acetabular cup, the radial flange having timing members to seat the adaptor to the acetabular cup.

16. A prosthesis impactor assembly for inserting an acetabular cup comprising:
an impactor handle having a receiving opening;
an adaptor that is separate from the impactor handle, the adaptor having a first end and a second end, wherein an impact head is provided at the first end of the adaptor, wherein an extension is provided at the second end of the adaptor to be received in the receiving opening of the impactor handle, and wherein the adaptor has a central bore that extends from the first end to the second end of the adaptor; and,
a connector rotatably secured within the central bore of the adaptor, the connector having a first end and a second end, wherein the first end of the connector has a prosthesis connector member for connecting to the acetabular cup, wherein the connector member extends out of the central bore of the adaptor to engage the acetabular cup, and wherein the second end of the connector has a drive connector.

17. The prosthesis impactor assembly of claim 16, further comprising a locking mechanism connected to the impactor handle to removably secure the adaptor and connector to the impactor handle, the locking mechanism comprising a locking blade, a pivotable locking lever, and a locking spring, wherein the locking blade moves linearly from a first position to a second position, the locking blade engaging the adaptor in the first position to axially lock the adaptor in the impactor handle, and the adaptor being removable from the impactor handle when the locking blade is in the second position.

18. The prosthesis impactor assembly of claim 16, further comprising a drive mechanism extending through a portion of the impactor handle and selectively engaging the drive connector of the connector to rotate the connector.

19. The prosthesis impactor assembly of claim 16, further comprising a plurality of tapered rib timing members on an exterior surface of the impactor handle, and mating tapered rib timing members on a mating surface of the adaptor for rotationally aligning the adaptor to the impactor handle.

20. A prosthesis impactor assembly for inserting an acetabular cup comprising:
an impactor handle having a receiver and a plurality of timing members adjacent the receiver;
an adaptor that is separate from the impactor handle, the adaptor having a first end and a second end, wherein an impact head is provided at the first end of the adaptor, wherein the second end of the adaptor is received by the receiving of the impactor handle, and wherein the adaptor has a central bore that extends from the first end to the second end of the adaptor, the adaptor further having a plurality of mating timing members to mate with the timing members of the impactor handle;
a connector rotatably secured within the central bore of the adaptor, the connector having a first end and a second end, wherein the first end of the connector has a prosthesis connector member for connecting to the acetabular cup, wherein the connector member extends out of the central bore of the adaptor to engage the acetabular cup, and wherein the second end of the connector has a drive connector; and,
a locking mechanism connected to the impactor handle to removably secure the adaptor and connector to the impactor handle, the locking mechanism comprising a locking blade that engages the adaptor in a first position of the locking blade to axially lock the adaptor in the impactor handle, and the adaptor being removable from the impactor handle when the locking blade is in a second position.
